(19) United States
(12) Patent Application Publication
(10) Pub. No.: US 2010/0030084 A1
(43) Pub. Date: Feb. 4, 2010

(54) DIFFUSE OPTICAL TOMOGRAPHY WITH MARKERS CONTAINING FLUORESCENT MATERIAL

(75) Inventors: Andy Ziegler, Wellington (NZ); Thomas Koehler, Norderstedt (DE); Tim Nielsen, Hamburg (DE); Martinus Bernardus Van Der Mark, Eindhoven (NL); Michael Cornelis Van Beek, Eindhoven (NL); Levinus Pieter Bakker, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/514,589

(22) PCT Filed: Nov. 12, 2007
(86) PCT No.: PCT/IB07/54593

§ 371 Date: May 13, 2009

(30) Foreign Application Priority Data
Nov. 17, 2006 (EP) 06124351.5

(51) Int. Cl. A61B 6/00; G06K 9/00

(52) U.S. Cl. 600/476; 382/131

(57) ABSTRACT

The invention relates to a system, a medical image acquisition system, and a method for imaging an interior of a turbid medium (25). The invention also relates to a marker (60) for use in the method for imaging an interior of a turbid medium (25). The system, the medical image acquisition system, and the method may be used for obtaining an image of an interior of a turbid medium (25) by: accommodation of a turbid medium (25) inside a receiving volume (20); irradiation of the receiving volume (20) with light from a light source; detection of light emanating from the receiving volume (20) as a result of irradiating the receiving volume (20) with light from the light source through the use of a photodetector unit. The detected light is then used to reconstruct an image of an interior of the turbid medium (25). According to the invention, the system, the medical acquisition system, and the method are adapted such that during a measurement the receiving volume (20) comprises at least one marker (60) comprising a predetermined concentration of a chosen fluorescent agent. The light source is arranged for generating excitation light that causes fluorescent emission in the marker (60) and the photodetector is arranged to detect light emanating from the receiving volume (20) as a result of irradiating the receiving volume (20) with excitation light. The use of a marker (60) according to the invention enables obtaining information relating to the geometry of the turbid medium (25). If the turbid medium (25) comprises an unknown concentration of a second fluorescent agent, and the light source and the photodetector unit are arranged for causing fluorescence in the second fluorescent agent and detecting the resulting fluorescence light, respectively, the use of a marker (60) according to the invention enables calibration of the signal resulting from this fluorescence light.

### FIELD OF THE INVENTION

The invention relates to a system for imaging an interior of a turbid medium, comprising:
a. a receiving volume for accommodating the turbid medium;
b. a light source for irradiating the receiving volume;
c. a photodetector unit for detecting light emanating from the receiving volume as a result of irradiating the receiving volume with light from the light source.

The invention also relates to a medical image acquisition system for imaging an interior of a turbid medium, comprising:
a. a receiving volume for accommodating the turbid medium;
b. a light source for irradiating the receiving volume;
c. a photodetector unit for detecting light emanating from the receiving volume as a result of irradiating the receiving volume with light from the light source.

The invention also relates to a method for imaging an interior of a turbid medium, comprising the following steps:
a. accommodation of the turbid medium inside a receiving volume;
b. irradiation of the receiving volume with light from a light source;
c. detection of light emanating from the receiving volume as a result of irradiating the receiving volume with light from the light source, through use of a photodetector unit.

The invention also relates to a marker for use in:
a. a system for imaging an interior of a turbid medium;
b. a medical image acquisition system for imaging an interior of a turbid medium;
c. a method for imaging an interior of a turbid medium.

### BACKGROUND OF THE INVENTION

An embodiment of a system and method of this kind is known from U.S. Pat. No. 6,327,488B1. The described system and method can be used for imaging an interior of a turbid medium, such as biological tissue. In medical diagnostics the system and method may be used for imaging, for instance, an interior of a female breast. The receiving volume receives a turbid medium, such as a breast. Next, light from the light source is coupled into the receiving volume. This light is chosen such that it propagates through the turbid medium. This procedure is called transillumination. Light emanating from the receiving volume as a result of irradiating the receiving volume with light from the light source is detected through the use of a photodetector unit. Based on the detected light, an image of an interior of the turbid medium is then reconstructed.

It is a characteristic of the known system and method that the geometry of the turbid medium cannot be easily determined.

### SUMMARY OF THE INVENTION

It is an object of the invention to provide the system with an easy means for obtaining information relating to the geometry of the turbid medium. According to the invention this object is achieved in that the system is arranged such that:
the system further comprises at least one marker for use in the receiving volume, comprising a predetermined concentration of a chosen first fluorescent agent;
the light source is arranged for irradiating the receiving volume with first excitation light, the first excitation light being chosen for causing fluorescent emission in the first fluorescent agent;
the photodetector unit is arranged for detecting light emanating from the receiving volume as a result of irradiating the receiving volume with first excitation light from the light source.

The invention is based on the recognition that detecting fluorescence light from at least one marker comprising a predetermined concentration of a chosen first fluorescent agent, with the at least one marker being located on the surface of a turbid medium or surrounding a turbid medium comprised in the receiving volume, enables obtaining information relating to the geometry of the turbid medium. If the at least one marker is located on the surface of the turbid medium, the origin of the fluorescence light emanating from the at least one marker indicates the surface of the turbid medium. If the at least one marker closely surrounds the turbid medium comprised in the receiving volume, determining the regions comprised in the receiving volume from which fluorescence light does and does not emanate indicates the volume occupied by the turbid medium and the geometry of that volume. The predetermined concentration of a chosen first fluorescent agent comprised in the at least one marker must be such that a fluorescent signal can be obtained from the at least one marker.

An embodiment of the system according to the invention is characterized in that a plurality of markers is located at predetermined positions on the surface of the turbid medium. This embodiment has the advantage that it is easy to implement as it only requires a plurality of markers to be attached to the surface of the turbid medium. In medical diagnostics where the system may be used for, for example, imaging an interior of a female breast, a plurality of markers may be temporarily glued to the skin using an easily removable glue.

A further embodiment of the system according to the invention is characterized in that at least one marker is arranged for surrounding the turbid medium. The space inside the receiving volume not occupied by the turbid medium may be filled with a medium. If this medium comprises a predetermined concentration of a fluorescent agent, then the medium constitutes a marker in accordance with the invention. A special case of this embodiment is the case in which the space inside the receiving volume not occupied by the turbid medium comprises an optical adaptation medium. In the known system the space inside the receiving volume not occupied by the turbid medium comprises an optical adaptation medium for reducing the effect on the image reconstruction process of optical boundary effects stemming from coupling light into and out of the turbid medium. According to the invention the adaptation medium can play a second role, in addition to the role of optical adaptation medium, by comprising a predetermined concentration of a fluorescent agent. This has the advantage of easy implementation, as the adaptation medium already present in the known system is used. Additionally, determining the amount of fluorescence light emanating from the marker at a plurality of positions relative to the turbid medium, in combination with the known geometry of the receiving volume, gives information relating to the geometry of the turbid medium, as the amount of fluorescence light is determined by the amount of marker between the boundary of the receiving volume facing the turbid medium and the exterior of the turbid medium itself.

A further embodiment of the system according to the invention is characterized in that the turbid medium comprises a second fluorescent agent, in that the light source is arranged for irradiating the receiving volume with second excitation light, the second excitation light being chosen for causing fluorescence emission in the second fluorescent agent, and in that the photodetector unit is arranged for detecting light emanating from the receiving volume as a result of irradiating the receiving volume with second excitation light. This embodiment has the advantage that the use of a marker according to the invention provides an easy means for calibrating the fluorescence signal stemming from the fluorescence emission from the second fluorescent agent comprised in the turbid medium. As described hereinabove, an embodiment of the system is known from U.S. Pat. No. 6,327,488B1 in which the system is used for transillumination measurements. As an alternative, however, the light source may be arranged to generate excitation light chosen for causing fluorescent emission in a fluorescent agent comprised in the turbid medium. In that case, the photodetector unit is arranged for detecting light emanating from the receiving volume as a result of irradiating the receiving volume with excitation light. Analogous to a transillumination measurement, light emanating from the receiving volume as a result of irradiating the receiving volume with light from the light source is detected through the use of a photodetector unit. Based on the detected light, an image of an interior of the turbid medium is then reconstructed. This procedure is called a fluorescence measurement and is described in European patent application with application number 05111164.9 (PH004270, attorney reference). If used for fluorescence measurements, it is a characteristic of the known system and method that a number of effects exist that disturb the reconstruction of an absolute scale of the fluorescence signal detected using the photodetector unit. For example, an optical filter intended to prevent excitation light from reaching the photodetector unit does not suppress the excitation light completely. Thus, a remaining part of excitation light measured when measuring the fluorescence signal impedes an effective calibration of the fluorescence signal. In a fluorescence measurement it is an additional advantage of the invention that the use of a marker comprising a predetermined concentration of a chosen first fluorescent agent provides an easy means for calibrating a fluorescence signal collected from the turbid medium. The additional advantage is based on the recognition that collecting a first fluorescence signal from a marker comprised in the receiving volume and comprising a predetermined concentration of a chosen first fluorescent agent enables the calibration of a second fluorescence signal collected from a turbid medium also comprised in the receiving volume and comprising an unknown concentration of a second fluorescent agent. For the purpose of calibration, the first fluorescent agent comprised in the at least one marker according to the invention and the second fluorescent agent comprised in the turbid medium are preferably the same, because then the signals from the first and second fluorescent agent can be easily compared. As the number of markers used increases, the accuracy of the calibration also increases. To obtain information relating to the geometry of the turbid medium, the first fluorescent agent comprised in the at least one marker according to the invention and the second fluorescent agent comprised in the turbid medium are preferably different. In that case signals stemming from the different agents are easier to distinguish, making it easier to determine the exterior of the turbid medium.

The object of the invention is also achieved in that the medical image acquisition system for imaging an interior of a turbid medium is arranged such that:
the system further comprises at least one marker for use in the receiving volume, comprising a predetermined concentration of a chosen first fluorescent agent;
the light source is arranged for irradiating the receiving volume with first excitation light, the first excitation light being chosen for causing fluorescent emission in the first fluorescent agent;
the photodetector unit is arranged for detecting light emanating from the receiving volume as a result of irradiating the receiving volume with first excitation light from the light source. A medical image acquisition system for imaging an interior of a turbid medium benefits from any of the previous embodiments.

The object of the invention is also achieved in that the method for imaging an interior of a turbid medium is arranged such that:
in the step of irradiation of the receiving volume, the receiving volume comprises at least one marker comprising a predetermined concentration of a chosen first fluorescent agent;
the light from the light source is chosen for causing fluorescent emission in the first fluorescent agent;
in the step of detection of light emanating from the receiving volume, the photodetector unit is arranged for detecting light emanating from the receiving volume as a result of irradiating the receiving volume with light chosen for causing fluorescent emission in the first fluorescent agent.

A method for imaging an interior of a turbid medium benefits from any of the previous embodiments.

The object of the invention is further achieved by means of a marker for use in a method for imaging an interior of a turbid medium, the marker comprising a predetermined concentration of a chosen fluorescent agent.

### BRIEF DESCRIPTION OF THE DRAWINGS

These and other aspects of the invention will be further elucidated and described with reference to the drawings, in which:

FIG. 1 schematically shows an embodiment of a system for imaging an interior of a turbid medium according to the invention;

FIG. 2 schematically shows a plurality of fluorescent markers located on the surface of a turbid medium, in this case a female breast;

FIG. 3 schematically shows an embodiment of a medical image acquisition system according to the invention.

### DETAILED DESCRIPTION OF EMBODIMENTS

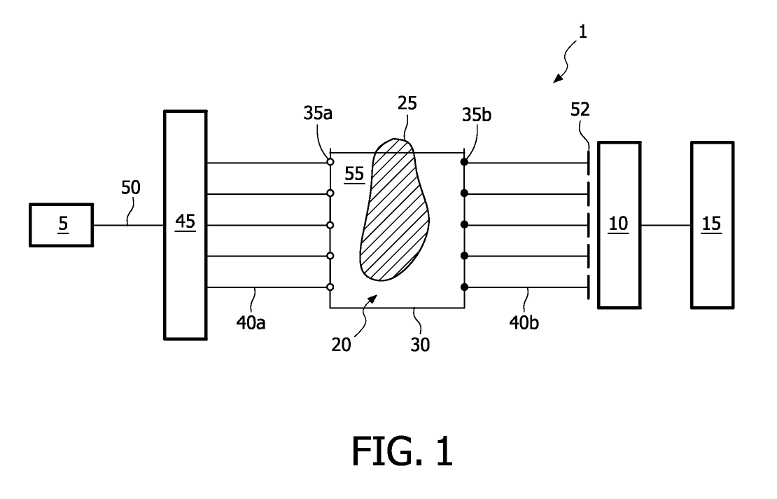

FIG. 1 schematically shows an embodiment of a system for imaging an interior of a turbid medium according to the invention. The system 1 comprises a light source 5 for emitting excitation light, the excitation light being chosen such that it causes fluorescent emission in a fluorescent agent in a turbid medium 25, a receiving volume 20 for receiving the turbid medium 25, said receiving volume 20 being bound by a receptacle 30, said receptacle comprising a plurality of entrance positions for light 35a and exit positions for light 35b, and light guides 40a and 40b coupled to said entrance positions for light 35a and exit positions for light 35b, respectively. The system 1 further comprises a photodetector unit 10 for detecting light emanating from the receiving volume 20, an image reconstruction unit 15 for deriving an image of an interior of the turbid medium 25, based on light detected using the photodetector unit 10, and a selection unit 45 for coupling the input light guide 50 to a number of selected entrance positions for light 35a in the receptacle. For the sake of clarity, entrance positions for light 35a and exit positions for light 35b have been positioned at opposite sides of the receptacle 30. In reality, however, they may be distributed around the receiving volume 20. The receiving volume 20 receives a turbid medium 25. The turbid medium 25 is then irradiated with light from the light source 5 from a plurality of positions by coupling the light source 5 to successively selected entrance positions for light 35a, using the selection unit 45. The light is chosen such that it is capable of propagating through the turbid medium 25. If, as may be the case in medical diagnostics, the system 1 is used for imaging an interior of a female breast, suitable light is, for instance, laser light with a wavelength within the range of 400 nm to 1400 nm. Light emanating from the receiving volume 20 as a result of irradiating the turbid medium 25 is detected from a plurality of exit positions, using exit positions 35b and using the photodetector unit 10. The detected light is then used to reconstruct an image of an interior of the turbid medium 25. To reduce the effect on the image reconstruction process of optical boundary effects resulting from coupling light into and out of the turbid medium 25, the turbid medium 25 is surrounded in the known system by an adaptation medium 55. This adaptation medium has optical characteristics, such as an absorption coefficient, chosen such that they match the optical characteristics of the turbid medium 25. If the system 1 is used for fluorescence measurements, the light source 5 is arranged to generate excitation light chosen such that it causes fluorescent emission in a fluorescent agent comprised in the turbid medium 25. The system 1 must then be arranged such that excitation light emanating from the receiving volume 20 can be distinguished from fluorescence light emanating from the receiving volume 20. This can be achieved, for instance, by arranging the system 1 such that light emanating from the receiving volume 20 passes through an optical filter 52 that filters out excitation light. However, the optical filter 52 does not suppress the excitation light completely. Hence, a true calibration of a signal resulting from detected fluorescence light is hampered. In an embodiment of the system 1 according to the invention, the adaptation medium 55 may become a marker according to the invention by comprising a predetermined concentration of a chosen fluorescent agent. By determining the amount of fluorescence light emanating from the receiving volume 20 from a plurality of positions relative to the turbid medium 25, information relating to the geometry of the turbid medium 25 can be obtained. As the amount of fluorescence light emanating from a unit of volume of the adaptation medium 55 comprising a predetermined concentration of a chosen fluorescent agent is known, as is the geometry of the receiving volume 20, the geometry of the turbid medium 25 can be deduced from the amount of fluorescence light emanating from the receiving volume 20. The information relating to the geometry of the turbid medium 25 can then be used during the process of reconstructing an image of an interior of the turbid medium 25. An adaptation medium 55 used as a marker according to the invention is a special case of the embodiment of the invention in which at least one marker is arranged for surrounding the turbid medium 25 comprised in the receiving volume 20. Such a marker need not play the role of an adaptation medium, although this is an option as explained above.

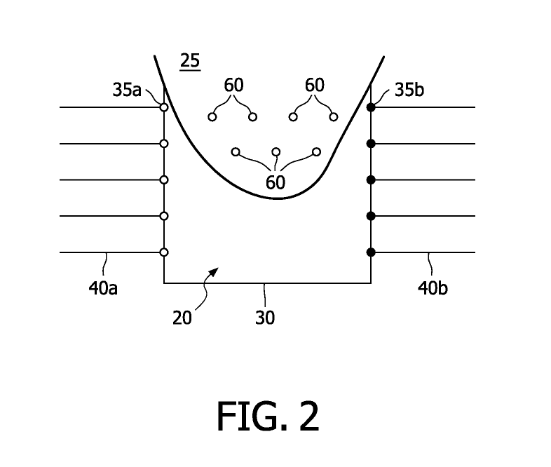

FIG. 2 schematically shows a plurality of fluorescent markers 60 located on the surface of a turbid medium 25, in this case a female breast. The breast is comprised in a receiving volume 20, the receiving volume 20 being bound by a receptacle 30 as shown in FIG. 1. In FIG. 2 the markers 60 are shown as circular objects comprising a predetermined concentration of a chosen fluorescent agent. However, the precise shape of the markers 60 is not essential. For calibration purposes, the size of the markers 60 should be larger than the resolution of the imaging process. The resolution of the imaging process may be different at different positions in the receiving volume 20. When imaging biological tissue, for instance, the imaging resolution may vary depending on the position in the tissue. In optical tomography employing markers 60 having a size larger than the resolution of the imaging process means, in this case, that the size of the markers 60 should be in the order of 1 to 2 cm. However, if the markers 60 are used to obtain information relating to the geometry of the turbid medium 25, the precise size of the markers 60 is not crucially important as long as the fluorescent emission from the markers 60 can be detected. The plurality of markers 60 has been evenly distributed over the surface of the breast to facilitate obtaining information relating to the geometry of the breast by detecting the fluorescent markers 60. To obtain information relating to the geometry of the breast, the fluorescent agent comprised in the markers 60 is preferably different from the fluorescent agent comprised in the turbid medium 25. In that case the fluorescence signal containing information relating to the geometry of the turbid medium 25 can be easily distinguished from the fluorescence signal from the fluorescent agent comprised in the turbid medium 25. If different fluorescent agents are used, light source 5 (see FIG. 1) must be arranged such that it can generate first excitation light and second excitation light, with the first excitation light causing fluorescent emission in the fluorescent agent comprised in the markers 60, and the second excitation light causing fluorescent emission in the fluorescent agent comprised in the turbid medium 25. The markers 60 can be attached to the breast by temporarily gluing them to the skin, using an easily removable glue. Clearly, a fluorescent marker according to any one of the embodiments of the invention may be used to obtain information relating to the geometry of a turbid medium 25, without the turbid medium 25 necessarily comprising a fluorescent agent itself.

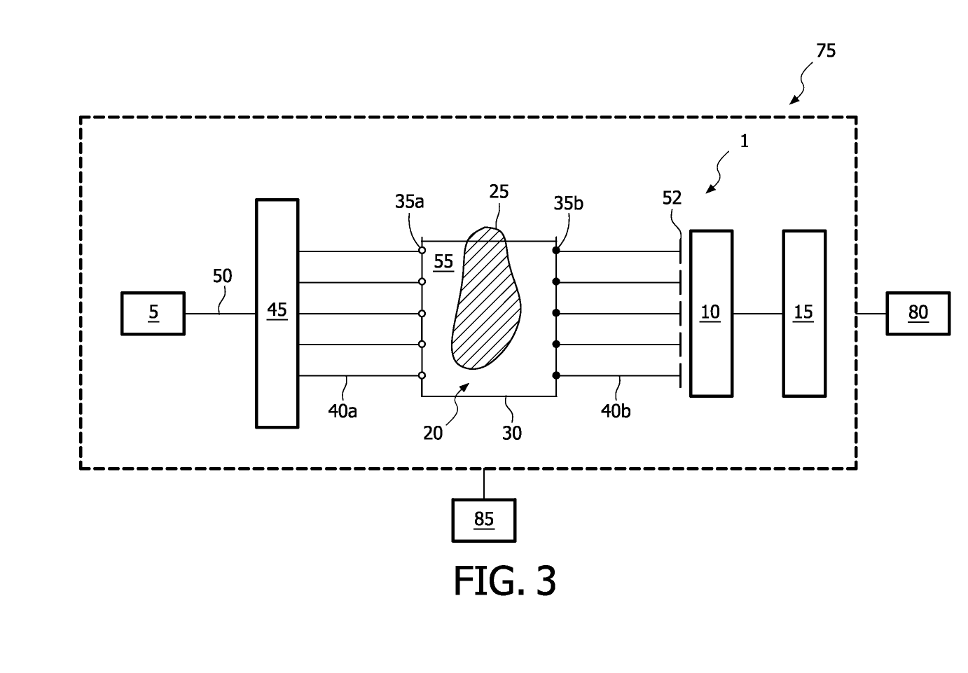

FIG. 3 schematically shows an embodiment of a medical image acquisition system according to the invention. The medical image acquisition system 75 comprises the system 1 discussed in FIG. 1, indicated by the dashed square. In addition to the system 1, the medical image acquisition system 75 further comprises a screen 80 for displaying an image of an interior of the turbid medium 25 reconstructed by the image reconstruction unit 15 and an input interface 85, for instance, a keyboard enabling an operator to interact with the medical image acquisition system 75.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A system (1) for imaging an interior of a turbid medium (25), comprising:
a. a receiving volume (20) for accommodating the turbid medium (25);
b. at least one marker (55, 60) for use in the receiving volume (20), the at least one marker (55, 60) comprising a predetermined concentration of a chosen first fluorescent agent;
c. a light source (5) for irradiating the receiving volume (20) with first excitation light, the first excitation light being chosen for causing fluorescent emission in the first fluorescent agent;
d. a photodetector unit (10) for detecting light emanating from the receiving volume (20) as a result of irradiating the receiving volume (20) with first excitation light from the light source (5).

2. A system (1) as claimed in claim 1, wherein a plurality of markers (55, 60) is located at predetermined positions on the surface of the turbid medium (25).

3. A system (1) as claimed in claim 1, wherein at least one marker (55, 60) is arranged for surrounding the turbid medium (25).

4. A system (1) as claimed in claim 1, wherein the turbid medium (25) comprises a second fluorescent agent, the light source (5) is arranged for irradiating the receiving volume (20) with second excitation light, the second excitation light being chosen for causing fluorescent emission in the second fluorescent agent, and the photodetector unit (10) is arranged for detecting light emanating from the receiving volume (20) as a result of irradiating the receiving volume (20) with second excitation light.

5. A medical image acquisition system (75) for imaging an interior of a turbid medium (25), comprising:
a. a receiving volume (20) for accommodating the turbid medium (25);
b. at least one marker (55, 60) for use in the receiving volume (20), the at least one marker (55, 60) comprising a predetermined concentration of a chosen first fluorescent agent;
c. a light source (5) for irradiating the receiving volume (20) with first excitation light, the first excitation light being chosen for causing fluorescent emission in the first fluorescent agent;
d. a photodetector unit (10) for detecting light emanating from the receiving volume (20) as a result of irradiating the receiving volume (20) with first excitation light from the light source (5).

6. A method for imaging an interior of a turbid medium (25), comprising the following steps:
a. accommodation of the turbid medium (25) inside a receiving volume (20);
b. irradiation of the receiving volume (20) with light from a light source (5), the receiving volume (20) comprising at least one marker (55, 60) comprising a predetermined concentration of a chosen first fluorescent agent, and the light from the light source (5) being chosen for causing fluorescent emission in the first fluorescent agent;
c. detection of light emanating from the receiving volume (20) as a result of irradiating the receiving volume (20) with light chosen for causing fluorescent emission in the first fluorescent agent.

7. A marker (55, 60) for use in a method for imaging an interior of a turbid medium (25), the marker (55, 60) comprising a predetermined concentration of a chosen fluorescent agent according to claim 6.
